(19) United States
(12) Patent Application Publication
(10) Pub. No.: US 2008/0267479 A1
(43) Pub. Date: Oct. 30, 2008

(54) Faster Rates for Real-Time 3D Volume Rendered Images

(75) Inventor: James Jago, Seattle, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/097,304

(22) PCT Filed: Dec. 8, 2006
(86) PCT No.: PCT/IB06/54722

§ 371 Date: Jun. 13, 2008

Related U.S. Application Data
(60) Provisional application No. 60/750,752, filed on Dec. 15, 2005.

(51) Int. Cl. A61B 8/00

(52) U.S. Cl. 382/131

(57) ABSTRACT

The present invention provides for generating ultrasound volume images at a higher rate by generating rendered images at the same rate as that of the acquired frames.

The present invention relates to generating ultrasound volume rendered images at a higher rate than that at which the underlying 3 D ultrasound data is acquired. In particular, the present invention relates to generating volume rendered images at higher rates by incorporating new ultrasound data into the 3 D data set as soon as it is acquired and re-projecting at higher rates.

Three dimensional ultrasound imaging, both single sweep (3 D) and real-time (commonly known as 4 D or Live 3 D), is becoming more and more prevalent on modern ultrasound systems. Clinically, it is used in many applications including: OB (for example for baby faces and for diagnosis of congenital defects, Cardiac (for example for quantitative assessment of ejection fraction and for visualization of cardiac function), and others.

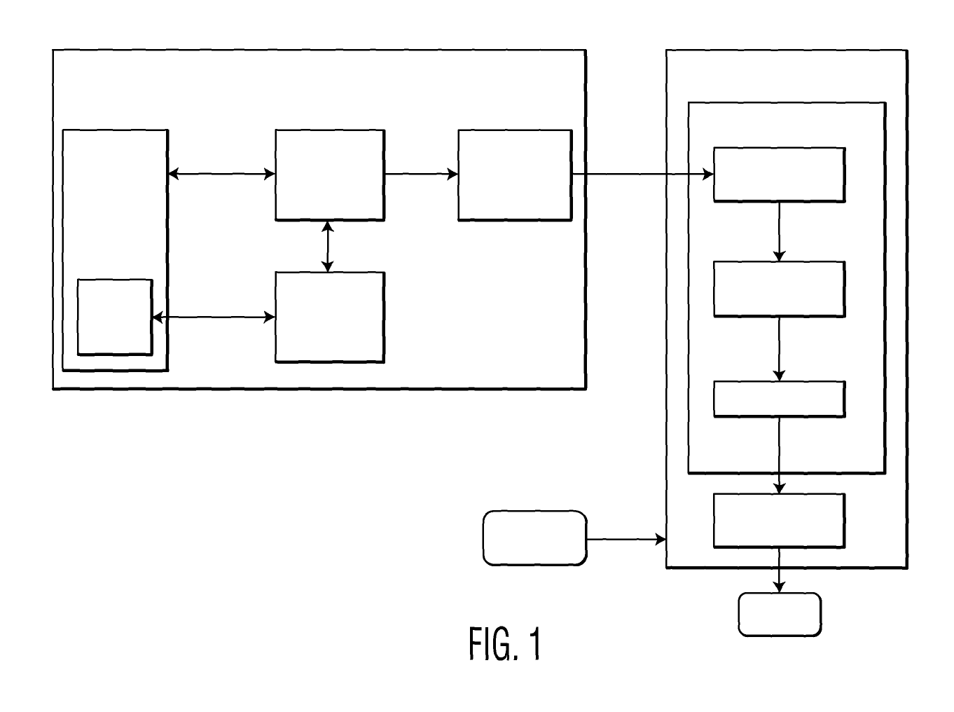

Real-time (Live) 3 D involves acquisition and display of a full volume of data at a rate fast enough for the 3 D display to show 3 D rendered images or multiple slices at a clinically useful rate. Capture of 3 D data for general imaging applications is done using motorized or 2 D array transducers. FIG. 1 shows a block diagram of a typical 3 D/4 D data path for a motorized transducer, showing both an acquisition step and a visualization step. The signal path for a non-motorized 2 D array transducer is similar, but the motor controller and gear train are not used.

Ultrasound volume rendered images are generated by projecting a 3 D data set onto a 2 D surface. These images are typically generated at the same rate at which the underlying 3 D ultrasound data is acquired, which is limited by acoustic propagation time and/or (for mechanical 3 D probes) mechanical limitations. Most clinicians would prefer these rates to be higher.

Motorized acquisition is done by mechanically moving a ID array under control of the Motor Controller and acquiring beam data. The probe is moved continuously and scan lines (beams) from the entire volume or only at sites where multiple 2 D slice views are desired are acquired during rotation or translation. The focal delays, weights and timing for these beams is set by the Front End Controller. Acquisition using 2 D array transducers (X-Matrix) is done by steering beams electronically in both azimuth and elevation, again under the control of the Front End Controller and typically, also a micro-beam former within the 2 D transducer itself. The RF beams so formed are then fed through a Signal Conditioning module, which typically performs various standard ultrasound signal processing operations such as envelope detection, compression, etc.

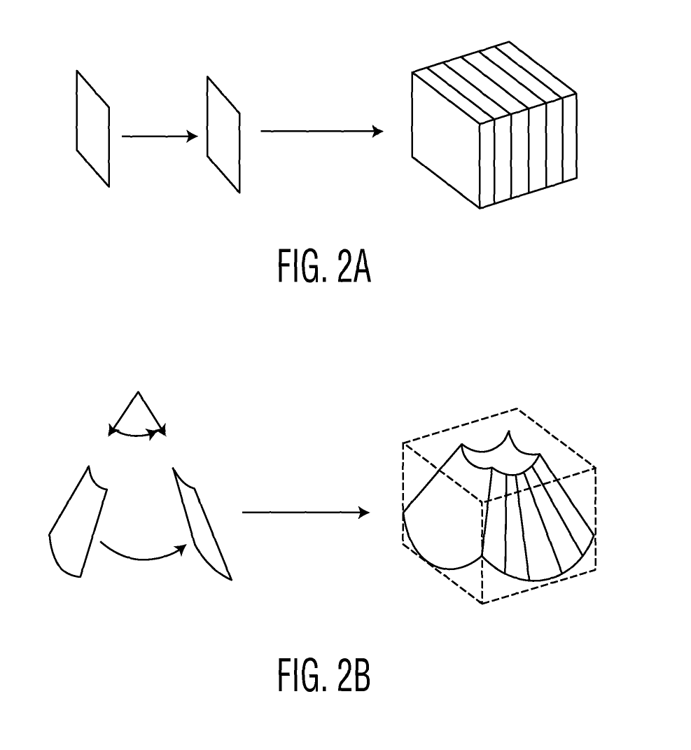

The scan converter of the visualization software generates the volume or slice view frames by assembling the scan lines by position, as shown in FIG. 2 for both linear and sector formats. The full volumes are kept in a 3 D Cineloop history buffer in Image Memory, either to be transferred directly for real-time (live 3 D) rendering or can be saved and restored for later 3 D review.

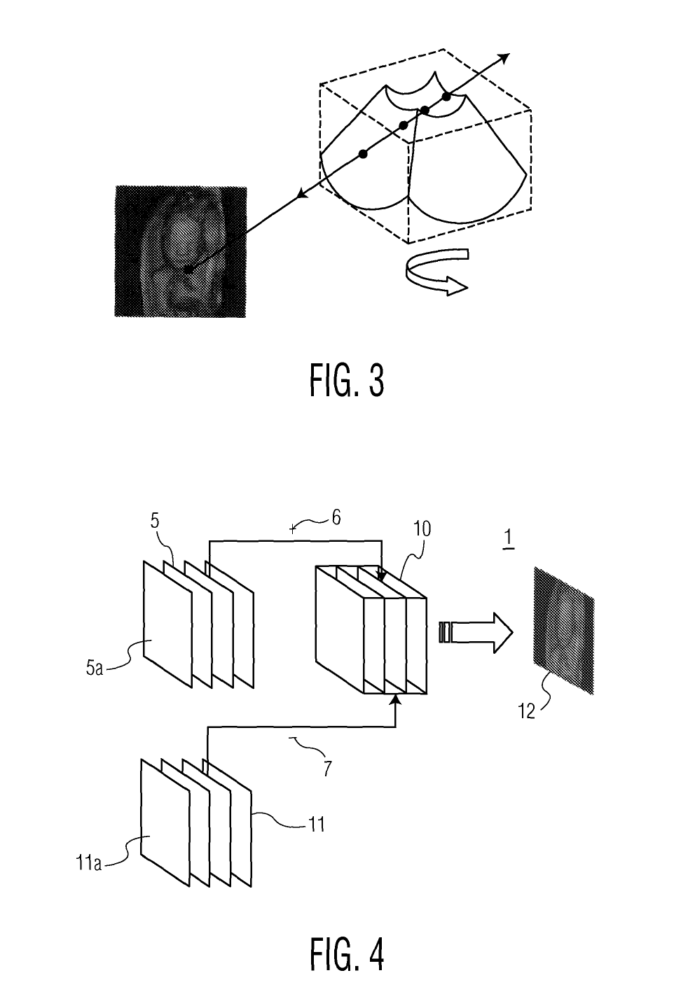

In most cases ultrasound 3 D or 4 D views, known as rendered views, are generated by projecting the entire volume of data along rays in the direction of a viewpoint onto a 2 D plane. Controls can be manipulated to adjust the viewpoint direction, transparency and texture of the volume as well as trim and sculpt away outer regions to better vie interior regions. The result is a “3 D image”, which provides qualitative visualization of the volume. While specific implementations differ, volume rendering approximates the propagation of light (or ultrasound) through a semi-opaque volume. The basic steps of all volume-rendering algorithms consist of assigning colors and opacities to each sample in the volume projecting the samples along linear rays to a 2 D image, and accumulating the samples projected along each ray. This process is shown in FIG. 3 below, for a single viewing ray and associated image pixel.

One limitation of existing ultrasound systems operating in real time 3 D is that the volume rendered images are typically generated at the same rate at which the underlying 3 D ultrasound data is acquired—i.e. the visualization rate is the same as the acquisition rate. For a large field view (especially in OB and General Imaging) and for an acceptable image quality, a very large number of acoustic scan lines must be acquired in order to adequately sample the volume, resulting in acquisition rates that may be as low as a few Hz. This is true even for a Matrix (i.e. 2 D) array. Since the visualization rate is the same as the acquisition rate, this creates a problem for the user who is trying to interact, in real-time, with the anatomy being visualized. One way to improve the volume rates is to acquire less data, but this sacrifices either field of view or image quality or both.

It would be desirable to provide ultrasound volume images generated at a higher rate that avoids the drawbacks of the aforementioned prior art.

Real-time spatial compounding (known as SonoCT at Philips), which involves averaging ultrasound data obtained from multiple, overlapping 2 D images acquired from different angles, has a similar problem in that a large amount of acoustic data is required to generate one complete compounded image, so in effect the compounded frame rate is low. However, experience from SonoCT has shown that the user experience is much improved if the compounded images are updated as soon as any new information arrives—specifically, by updating the compounded image each time a new component frame (i.e. one steering angle) is acquired, as opposed to waiting for an entire compound sequence (see U.S. Pat. No. 6,126,599). Essentially, we are presenting the compounded images at the component frame rate instead of the compound frame rate, and these are similar to the images that would be obtained if one could interpolate perfectly between the truly independent compounded images. The user typically perceives the frame rate to be about 2× the actual compounded rate. Another advantage is latency, since the user sees new information at the rate of the component frames instead of the fully compounded image.

Since volume projection is a very similar concept to the frame averaging used in SonoCT, these same benefits can be transferred to 3 D volume rendered imaging by updating the rendered image as each component 2 D slice is obtained, or at some other intermediate rate. The idea is to update the volume rendered image at a rate that is determined by clinical need and processing power, instead of at the 3 D volume acquisition rate.

The present invention provides for generating ultrasound volume images at a higher rate by generating rendered images at the same rate as that of the acquired 2 D frames rather than at the rate for the acquired 3 D volumes, or at some intermediate rate.

FIG. 1 illustrates a typical real time 3 D signal path showing real time 3 D acquisition;

FIG. 2a illustrates a conventional 3 D scan conversion for a linear sweep;

FIG. 2b illustrates a conventional 3 D scan conversion for a fan sweep;

FIG. 3 illustrates the conventional methodology for volume rendering; and

FIG. 4 illustrates how the present invention modifies a typical 3 D volume rendering so that rendered images are generated at the rate of the acquired 2 D frames, rather than at the rate of the acquired volumes.

Referring to the drawings, FIG. 4 describes the operation of the present invention. The system (1) requires that each 2 D frame (5a) takes a time (t) to acquire, so that a complete acquisition volume (5), consisting of N 2 D frames, is acquired in a time (Nt). In the prior art the 3 D scan converter would then generate 3 D volumes at the acquisition volume rate (1/Nt) and these would also be rendered (and hence visualized, 12) at the same rate (1/Nt). However as shown in FIG. 4, by continuously updating the scan converted volume with new image data as soon as it arrives, specifically by adding (6) the most recent 2 D frame (5 a) and subtracting (7) the equivalent 2 D frame (11a) from the previously acquired volume (11a), then it is possible to generate volume rendered images at the acquisition frame rate (1/t) instead of the volume rate (1/Nt).

Since Volume rendering at the 2 D acquisition frame rate results in rendered volumes that have much image data in common (only 1 out of N 2 D frames is unique), so that the rendered images look very similar, in practice it is more likely that volume rendering will occur at a rate somewhere between the 2 D acquisition rate (1/t) and the 3 D acquisition rate (1/Nt). Also, volume rendering at the 2 D acquisition rate may also exceed the system processing resources, since volume rendering is quite processing intensive. Experience from SonoCT has suggested that a volume rendering rate of around (2/Nt), i.e. twice the acquisition volume rate, may represent a good compromise.

This concept requires a 3 D volume buffer (10), as shown in FIG. 4, which will be used to accumulate the most recent volume data, and a separate 3 D volume buffer (11) which stores the previous volume date. New 2 D frames (5) will be added (6) to the buffer (10) and will replace older 2 D frames obtained from the same spatial location in the stored 3 D volume (11) which are subtracted (7) from this buffer (10). Volume rendering (12) will run on the 3 D volume buffer at the chosen rate—ie decoupled from the volume acquisition rate

Thus, the present invention provides a method and system for modifying the typical 3 D volume rendering (shown in FIG. 2b and FIG. 3) by generating rendered images at the same rate of the acquired frames (1/t) instead of at the rate of the acquired volumes as is typically done and illustrated in FIGS. 2b and 3. This operation is software implemented for an acquisition based on 2 D frame rate rather than the acquired volume rate.

One issue is the risk of “tears” between parts of the volume that have been acquired at different times. This can be mitigated by always projecting at or close to right angles to the 2 D sweep direction in which case any artifacts will be no worse than they would be in the projected views that would normally (i.e. at the acquisition volume rate) be displayed. On a Matrix array, this is easy to ensure for projections that are not directly along the beam axis since, in principle, 2 D slices can be swept in any orientation as long as the apex of the beams is at the transducer.

The present invention can run on any ultrasound system that supports real-time 3 D imaging and therefore the present invention is not limited to any one ultrasound system. By way of illustrative examples but not intended to be limiting, the present invention can run on the following ultrasound systems: Philips iU22; Philips iE33; GE Logic 9; GE Voluson; Siemens Antares; and Toshiba Aplio.

While presently preferred embodiments have been described for purposes of the disclosure, numerous changes in the arrangement of method steps and apparatus parts can be made by those skilled in the art. Such changes are encompassed within the spirit of the invention as defined by the appended claims.

1. A method for generating ultrasound volume rendered images the steps comprising:
generating 3 D volumes with a 3 D scan converter;
providing a 3 D volume buffer to accumulate recent volume data;
providing a 3 D volume buffer to store the previously acquired volume data,
continuously updating scan converted volume with new image data by adding recent 2 D frames and replacing older equivalent 2 D frames obtained from some spatial location in a 3 D volume in said buffer so that volume rendering runs on said buffer at a chosen rate, and ultrasound rendered images are generated at a higher rate than that at which an underlying 3 D ultrasound data is acquired.

2. The method according to claim 1 wherein said volume rendering occurs at a rate somewhere between a 2 D acquisition rate (1/t) and a 3 D acquisition rate (1/Nt).

3. The method according to claim 2 wherein said volume rendering rate is approximately twice said acquisition volume rate (2/Nt).

4. The method according to claim 1 further comprising:
projecting at or close to right angles to a 2 D sweep direction in order to prevent tears between parts of volume acquired at different times.

5. The method according to claim 1 wherein said buffer is software implemented.

6. A system for generating ultrasound volume rendered images comprising:
a 3 D scan converter for generating 3 D volumes;
a 3 D volume buffer for accumulating recent volume data;
said 3 D volume buffer receiving continuously updated scan converted volume with new image data by adding recent 2 D frames and replacing older equivalent 2 D frames obtained from a spatial location in a 3 D volume so that volume rendering in said buffer runs at a chosen rate and ultrasound rendered images are generated at a higher rate than that at which an underlying 3 D ultrasound data is acquired.

7. The system according to claim 6, wherein said volume rendering occurs at a rate somewhere between a 2 D acquisition rate (1/t) and a 3 D acquisition rate (1/Nt).

8. The system according to claim 7 wherein said volume rendering rate is approximately twice said acquisition volume rate (2/Nt).

9. The system according to claim 6 further comprising:
said system projects at or near right angles to a 2 D sweep direction in order to prevent tears between parts of volume acquired at different times.

10. The system according to claim 6 wherein said buffer is software implemented.
